(19) United States
(12) Patent Application Publication
(10) Pub. No.: US 2017/0158519 A1
(43) Pub. Date: Jun. 8, 2017

(54) ZEOLITE TYPE A SORBENT

(71) Applicant: NEOZEO AB, Stockholm (SE)

(72) Inventors: Petr VASILIEV, Sundbyberg (SE); Ocean CHEUNG, Täby (SE); Zoltan BACSIK, Sollentuna (SE); Hedin NIKLAS, Stockholm (SE)

(73) Assignee: NEOZEO AB, Stockholm (SE)

(21) Appl. No.: 15/115,552

(22) PCT Filed: Feb. 4, 2015
(86) PCT No.: PCT/SE2015/050122

§ 371 Date: Jul. 29, 2016

(30) Foreign Application Priority Data
Feb. 5, 2014 (SE) 1450120-9

(51) Int. Cl. C01B 39/14; B01J 20/18; B01D 53/02; C10L 3/10; C07C 7/13; C01B 39/02; B01J 20/30

(52) U.S. Cl. CPC C01B 39/14; C01B 39/026; B01J 20/186; B01J 20/3085; C10L 3/104; C07C 7/13; B01D 53/02; C10L 2290/542; B01D 2253/108; B01D 2256/245; B01D 2257/504

(57) ABSTRACT

The present invention relates to a zeolite type A, a method for preparing the zeolite type A, use of the zeolite type A for separating carbon dioxide from a composition comprising hydrocarbons, and a process for separating carbon dioxide from a gas composition using the zeolite type A. The zeolite type A comprising, based on total amount of exchangeable ions, less than 10% of potassium ions, less than about 10% of a second group of ions consisting of cesium ions, rubidium ions and mixtures thereof, and from about 80% up to about 90% of a third group of ions consisting of sodium ions, lithium ions and mixtures thereof

### FIELD OF INVENTION

The present invention relates to a zeolite type A, a method for preparing the zeolite type A, use of the zeolite type A for separating carbon dioxide from a composition comprising hydrocarbons, and a process for separating carbon dioxide from a gas composition using the zeolite type A.

### BACKGROUND OF THE INVENTION

In recent years, words like “global warming” and “greenhouse effect” have had their shares in the media world. It is in generally believed that global warming is the result of an increased concentration of the greenhouse gases in the atmosphere. Particularly in the last few decades, scientists and engineers have put a vast amount of efforts into controlling the levels of greenhouse gas emission through different means. One of the most well known topic is carbon capture and storage (CCS). CCS can be implemented by different means. Take a fossil fuel burning power plant as an example; carbon capture can be implemented as pre-combustion capture of CO₂, oxy-fuel combustion process, or post combustion capture of CO₂, as well as by other means. In the case of post combustion CO₂ capture, the exhaust gas from combustion (known as flue gas, consists mainly of N₂ but can have up to 15% CO₂) is passed through a process that removes CO₂ before being released into the atmosphere. This targets the release of CO₂ from point sources, and eliminates it.

Another process at which CO₂ needs to be removed from a stream of gas is bio-gas and natural gas upgrading. Regardless of its purpose, most modern internal combustion engines are designed to be fuel efficient and to have low emissions. These engines are designed to run on high quality fuel with very little contaminants. Fuel contaminates may include sulphur compounds in diesel, lead compounds in petrol (gasoline). Throughout the years, engine manufacturers and fuel companies have taken huge steps forward to produces ultra-low sulphur diesel, unleaded petrol fuel and extremely efficient fuel burning technologies to minimise the emission of harmful exhaust gases, particularly nitrogen oxides. In terms of bio-gas, which most often is produced from anaerobic digestion (AD) of man-made waste products, the presence of sulphur containing compounds, CO₂ and water is the main issue. AD biogas can contains up to 50% CO₂, depending on the production method. For fuel application, most liquefied natural gas (LNG) (or compressed natural gas—CNG) usually have at least 95% methane, but higher purity is preferred. Low impurity fuel has a lower energy per unit volume out, and will result in poor power output as well as high levels of harmful gases being emitted.

As one of the major impurities of bio-gas is CO₂, the removal of such a compound is very important. Currently, the process to remove CO₂ is often called as bio-gas upgrading. Bio-gas upgrading may be carried out using one of the following processes: water scrubbing, polyethylene glycol absorption, membrane separation, and pressure swing adsorption (PSA), vacuum swing adsorption (VSA) and temperature swing adsorption (TSA) The processes involving PSA/VSA/TSA require the gas to pass through an adsorbent, which can selectively adsorb one or more gas component, giving a stream of purified gas at the other end. For this process to work efficiently there must be a good adsorbent. Carbon molecular sieves (CMS) and activated carbons are both currently used in the commercialised processes. Whilst these carbon based materials offer good selectivity for CO₂, their CO₂ capacities allow room for the development of new sorbents.

U.S. Pat. No. 6,024,781 discloses methods of separating carbon dioxide from gaseous hydrocarbons using potassium modified 4A zeolite adsorbents.

U.S. Pat. No. 3,982,912 relates to a process for the preparation of a K-A type zeolite and separation of by adsorption of mixtures using the zeolite.

It would be advantageous to further improve the characteristics of zeolites, specifically type A zeolites, such as improved selectivity as to carbon dioxide and gaseous hydrocarbons. More specifically, it is an objective to further improve the selectivity of a type A zeolite for the separation of carbon dioxide from a methane containing gas composition.

### SUMMARY OF THE INVENTION

The present invention is directed to a novel zeolite type A comprising, based on total amount of exchangeable ions, less than 10% of potassium ions, less than about 10% of a second group of ions consisting of cesium ions, rubidium ions and mixtures thereof, and from about 80% up to about 90% of a third group of ions consisting of sodium ions, lithium ions and mixtures thereof.

The invention is further directed to a method for preparing a type A based on total amount of exchangeable ions, less than 10% of potassium ions, less than about 10% of a second group of ions consisting of cesium ions, rubidium ions and mixtures thereof, and from about 80% up to about 90% of a third group of ions consisting of sodium ions, lithium ions and mixtures thereof, comprising providing a zeolite type A structure and subjecting said zeolite type A zeolite to ion exchange.

The present invention is further directed to the use of a type A zeolite for separating carbon dioxide from a composition comprising hydrocarbons.

Finally, the invention is also directed to a process for separating carbon dioxide from a gas composition comprising hydrocarbons comprising contacting a type A zeolite type A comprising, based on total amount of exchangeable ions, less than 10% of potassium ions, less than about 10% of a second group of ions consisting of cesium ions, rubidium ions and mixtures thereof, and from about 80% up to about 90% of a third group of ions consisting of sodium ions, lithium ions and mixtures thereof, with the gas composition thereby forming a gas composition depleted in carbon dioxide.

### DETAILED DESCRIPTION OF THE INVENTION

It has been discovered that the adsorption and selectivity characteristics of a type A zeolite is significantly altered when exchanging the exchangeable cations. More specifically, the adsorption and selectivity is influenced by the type of cations and amounts.

Due to the presence of aluminia, zeolites exhibit a negatively charged framework, which is counter-balanced by positive cations. These cations can be exchanged by other cations to fine tune the pore size and hence adsorption characteristics. Any type A zeolite can be used for preparing the type A zeolites of the present invention. The sodium form of a zeolite type A can be used as precursor. The sodium form type A zeolite may be referred to as zeolite NaA, molecular sieve 4A or 4A zeolite.

According to the present invention the zeolite type A comprises, based on total amount of exchangeable ions, less than 10% of potassium ions, less than about 10% of a second group of ions consisting of cesium ions, rubidium ions and mixtures thereof, and from about 80% up to about 90% of a third group of ions consisting of sodium ions, lithium ions and mixtures thereof.

The zeolite type A of the present invention comprises potassium ions, a second group of ions consisting of cesium ions, rubidium ions and mixtures thereof, and a third group of ions consisting of sodium ions, lithium ions and mixtures thereof. Accordingly, the presence of potassium ions, a second group of ions, and a third group of ions in the zeolite type A of the invention is important. Hence, the language “less than” shall not be construed to mean that an indicated ion or group of ions is excluded from the zeolite type A. The lower range of the amount of ions, and group of ions can be from about 0.5%, such as from about 1%, from about 2%.

Zeolites contain framework ions (such as Al and Si) and extra-framework ions which are more easily exchangeable than the framework ions. Extra-frame ions may also be referred to as exchangeable ions.

All values related to the amount of ions are based on atomic % of extra-framework ions if not otherwise indicated.

The term about is herein contemplated to mean plus/minus 0.5%, suitably plus/minus 0.1%.

If not otherwise stated the amount of ions in the zeolite type A structure relates to the total amount of exchangeable ions.

The amount of exchangeable cations present in the type A zeolites of the present invention can be determined using energy dispersed X-ray spectroscopy (EDX) or with inductive coupled plasma—optical emission spectroscopy (ICP-OES).

The amount of potassium ions present in the zeolite A is preferably less than 10%, suitably less than about 9.5%, suitably less than about 9%, alternatively, preferably less than about 8.5% or 8%. The amount of potassium ions is preferably more than about 4%, preferably more than about 5%, more than about 6%, suitably more than about 6.5%, or, more than about 7%. Suitably, the amount of potassium ions present in the zeolite A is from about 5% up to about 10%, from about 6% up to about 10%, or from about 5% up to about 9%, preferably from about 6% up to about 8%, suitably from about 6% up to about 8%.

It should be understood that any upper limit can be combined with any lower limit. This is applicable to the other groups of ions.

The amount of the second group of ions consisting of cesium ions, rubidium ions and mixtures thereof present in the zeolite A is preferably from about 4% up to about 10%, preferably from about 5% up to about 10%, such as from about 6% up to about 10%, suitably from about 6% up to about 9%, such as from about 7% up to about 9%.

According to an embodiment, the second group of ions is chosen from cesium. Cesium is preferably present in the zeolite A in an amount of from about 6% to about 9.5%, preferably from about 6.5% up to about 9%, such as from about 6.5% up to about 8.5% suitably from about 7% to about 9, from about 7% up to about 8.5%, from about 7% up to about 8%.

According to another embodiment, the second group is chosen from rubidium. Rubidium is preferably present in the zeolite A in an amount of from about 4% up to about 10%, suitably from about 4 up to about 8%.

The zeolite A of the present invention comprises from about 80% up to about 90% of a third group of ions consisting of sodium and lithium. The third group of ions are preferably present in the zeolite A at an amount from about 82% up to about 88%.

According to an embodiment, the third group of ions is chosen from sodium.

According to a further embodiment the zeolite type A comprises from about 6% to 10% of potassium ions, from about 6% to about 10% of second group of ions consisting of cesium ions, rubidium ions and mixtures thereof, and from about 80% up to about 90% of a third group of ions consisting of sodium ions, lithium ions and mixtures hereof.

According to yet another embodiment, the zeolite type A comprises from about 6.5% to about 8% of potassium ions, from about 6% to about 8% of a second group of ions consisting of cesium ions, rubidium ions and mixtures thereof, and from about 80% up to about 90% of a third group of ions consisting of sodium ions, lithium ions and mixtures thereof.

According to yet another embodiment, the zeolite type A comprises from about 6.5% to about 8.5%, suitably from about 6.5% to about 8.0%, preferably from about 7% up to about 8%, of potassium ions, from about 6% to about 9%, from about 6% to about 9% preferably from about 7% up to about 9%, from about 7% up to about 8.5%, such as from about 7% up to about 8% of cesium ions, and from about 80% up to about 90%, preferably from about 82 up to about 88%, of sodium ions.

The cations of the zeolite A can be introduced by any suitable process, typically ion exchange.

One aspect of the invention relates to a method for preparing the zeolite type A comprising providing a zeolite type A structure and subjecting said zeolite type A zeolite to ion exchange.

The cations are introduced into the zeolite A by way of ion exchange. For example, a non-modified zeolite A, such as NaA zeolite (zeolite 4A) is brought to contact with potassium ions and second group ions and optionally third group ions in a solution or solutions, either simultaneously, or separately and successively to allow ion exchange of exchangeable cations in the type A zeolite. The cations may all be present in one solution, yet, preferably, one type of cations, e.g. potassium ions and second group ions (cesium and rubidium) are dissolved in different solutions. Preferably, the non-modified zeolite A is subjected sequentially to a solution comprising potassium ions and a solution comprising second group ions, and where appropriate, to a solution comprising third group ions. Preferably, the non-modified zeolite A is immersed in a solution comprising potassium ions before being immersed in a solution comprising ions of the second group. After each ion exchange procedure the zeolite is suitably washed and dried. When the starting zeolite is a sodium type A zeolite (NaA) and the objective is to prepare a zeolite A which does not comprise lithium ions, a certain amount of sodium ions are exchanged by potassium ions and second group ions. Such a zeolite A comprises potassium and second group ions, and the balance of exchangeable ions being sodium.

Another aspect of the invention relates to a process for separating carbon dioxide from a gas composition comprising hydrocarbons comprising contacting the zeolite type A with the gas composition thereby forming a gas composition depleted in carbon dioxide.

The carbon dioxide containing composition comprises hydrocarbons. The hydrocarbons may be saturated or unsaturated hydrocarbons such as alkanes, alkenes and alkynes. Typically the hydrocarbons comprise from 1 to 6 carbon atoms. Exemplified alkanes are methane, ethane, propane, butane, pentane and hexane. Typical alkenes are ethene, propene, butane, pentene and hexane. Representative alkynes are ethyne, such as acetylene, popyne, butyne and the like. The type A zeolite is particularly suited for the separation of carbon dioxide from a gas composition comprising alkanes, suitably methane.

The process for separating carbon dioxide from a gas composition comprising hydrocarbons using the the zeolite type A can be any suitable process. Exemplified processes can be various swing adsorption processes including pressure swing adsorption (PSA), temperature swing adsorption (TSA), vacuum swing adsorption (VSA) or any combination of swing adsorption processes.

The invention is further illustrated by the following examples which, however, are not intended to limit the same.

### Example

A highly crystalline and high quality zeolite A powder was used. Zeolite A sorbents containing Na⁺, K⁺ and Cs⁺ (samples A, B, C and D as evident from table 1) were obtained with a two steps partial ion exchange process. Here, the preparation steps of NaKCsA with approximately 7% K⁺, 7% Cs⁺ and 86% Na⁺ is given as an example. To prepare such zeolite sorbent, 2 g of zeolite NaA was mixed in 100 cm³ of a 0.01 mol/dm³ solution of KCl for 30 minutes at room temperature. The zeolite NaKA was then produced and was then separated from the solution, washed and dried at 373K for 2 hours. The produced NaKA was then mixed in 100 cm³ of a 0.01 mol/dm³ solution of CsCl for 30 minutes at room temperature. The final product was zeolite NaKCsA. The zeolite powder was separated from the solution, washed and dried at 373K for 2 hours. The procedures for producing zeolite NaKCsA with other cation compositions are detailed in supporting information

The cation composition of the ion exchanged zeolites were determined in-house using energy dispersed X-ray spectroscopy (EDX) and then further confirmed with inductive coupled plasma—optical emission spectroscopy (ICP-OES) by Medac Ltd, UK.

Gas adsorption (CO₂, N₂ and CH₄) isotherms of the zeolite sample were recorded on a Micromertrics ASAP2020 surface area analyser at 293K. The temperature of the analyses was controlled by circulation bath with a low molecular weight siloxane polymer. All samples were pre-treated/degassed by heating the sample to 623 K under high dynamic vacuum (1×10⁻⁴ Pa) for 6 hours.

TABLE 1
Elemental composition, equilibrium CH₄ and CO₂ uptake, and
selectivity of various NaKCsA. The selectivity is defined as follows:
selectivity (CO₂/CH₄) = uptake of CO₂ (at a partial pressure of 0.5 bar
of CO₂)/uptake of CH₄/(at a partial pressure of 0.5 bar of CH₄)/
(Partial pressure of CO₂/Partial pressure of CH₄)

| | | | | CO₂ | CH₄ | |
|---|---|---|---|---|---|---|
| Sample | Na at. % | Cs at. % | K at. % | (0.5 bar) | (0.5 bar) | Selectivity |
| A | 84.49 | 7.6 | 7.11 | 2.92 | 0.0025 | 1168 |
| B | 87.29 | 4.81 | 7.29 | 3.52 | 0.11 | 15 |
| C | 88.16 | 7.12 | 4.72 | 3.06 | 0.19 | 16 |
| D | 90.84 | 4.52 | 4.46 | 3.4 | 0.31 | 11 |

From table 1 it is evident that the amount of potassium and cesium has a significant impact on the selectivity. Sample A exhibit a significant improvement of the selectivity with regard to carbon dioxide and methane.

1. A zeolite type A comprising, based on total amount of exchangeable ions, less than 10% of potassium ions, less than about 10% of a second group of ions consisting of cesium ions, rubidium ions and mixtures thereof, and from about 80% up to about 90% of a third group of ions consisting of sodium ions, lithium ions and mixtures thereof.

2. The zeolite type A according to claim 1, comprising less than about 9% of potassium ions.

3. The zeolite type A according to claim 1, comprising less than about 8% of potassium ions.

4. The zeolite type A according to claim 1, comprising from about 6% to 10% of potassium ions, from about 6% to about 10% of second group of ions consisting of cesium ions, rubidium ions and mixtures thereof, and from about 80% up to about 90% of a third group of ions consisting of sodium ions, lithium ions and mixtures thereof.

5. The zeolite type A according to claim 1, wherein the second group of ions is cesium ions.

6. The zeolite type A according to claim 1, wherein the third group of ions is sodium ions.

7. A method for preparing the zeolite type A according to claim 1, comprising providing a zeolite type A structure and subjecting said zeolite type A zeolite to ion exchange.

8. Use of the zeolite type A according to claim 1 for separating carbon dioxide from a composition comprising hydrocarbons.

9. A process for separating carbon dioxide from a gas composition comprising hydrocarbons comprising contacting the zeolite type A according to claim 1 with the gas composition thereby forming a gas composition depleted in carbon dioxide.

10. The process according to claim 9, wherein the gas composition comprises methane.

11. The zeolite type A according to claim 2, comprising less than about 8% of potassium ions.

12. The zeolite type A according to claim 2, comprising from about 6% to 10% of potassium ions, from about 6% to about 10% of second group of ions consisting of cesium ions, rubidium ions and mixtures thereof, and from about 80% up to about 90% of a third group of ions consisting of sodium ions, lithium ions and mixtures thereof.

13. The zeolite type A according to claim 3, comprising from about 6% to 10% of potassium ions, from about 6% to about 10% of second group of ions consisting of cesium ions, rubidium ions and mixtures thereof, and from about 80% up to about 90% of a third group of ions consisting of sodium ions, lithium ions and mixtures thereof.

14. The zeolite type A according to claim 2, wherein the second group of ions is cesium ions.

15. The zeolite type A according to claim 3, wherein the second group of ions is cesium ions.

16. The zeolite type A according to claim 4, wherein the second group of ions is cesium ions.

17. The zeolite type A according to claim 2, wherein the third group of ions is sodium ions.

18. The zeolite type A according to claim 3, wherein the third group of ions is sodium ions.

19. The zeolite type A according to claim 4, wherein the third group of ions is sodium ions.

20. The zeolite type A according to claim 5, wherein the third group of ions is sodium ions.
